(19) United States
(12) Patent Application Publication
(10) Pub. No.: US 2023/0040329 A1
(43) Pub. Date: Feb. 9, 2023

(54) METHODS AND DEVICES FOR DELIVERING LIQUID THERAPEUTIC AGENTS IN SOLID TUMORS

(71) Applicant: JANSSEN PHARMACEUTICALS, INC., Titusville, NJ (US)

(72) Inventors: James A. FLEMING, Bethlehem, PA (US); Anthony R. DIUBALDI, Jackson, NJ (US); Rudolph CEDRO, JR., Stockton, NJ (US)

(21) Appl. No.: 17/783,815

(22) PCT Filed: Dec. 2, 2020
(86) PCT No.: PCT/IB2020/061410

§ 371 Date: Jun. 9, 2022

Related U.S. Application Data
(60) Provisional application No. 62/947,706, filed on Dec. 13, 2019.

(51) Int. Cl. A61M 25/00; A61M 25/04; A61M 25/06

(52) U.S. Cl. CPC A61M 25/0084; A61M 25/0074; A61M 25/04; A61M 25/0662; A61M 2025/0004; A61M 2025/0096

(57) ABSTRACT

Various exemplary methods and devices (10, 110, 210) for delivering liquid therapeutic agents in solid tumors are provided. In general, a delivery device configured to deliver a liquid therapeutic agent into a solid tumor or other soft tissue can be configured to compress tissue and seal any fluid gaps around the delivery device during the delivery of the liquid therapeutic agent. In an exemplary embodiment the delivery device includes three elongate tubular shafts (12, 14, 16, 112, 114, 116, 212, 214, 216) configured to move longitudinally relative to one another. The elongate tubular shafts are configured to cooperate with one another to deliver the liquid therapeutic agent through a passageway of the delivery device to the solid tumor or other soft tissue and seal any fluid gaps around the delivery device while the liquid therapeutic agent is delivered into the solid tumor or other soft tissue.

### CROSS REFERENCE TO RELATED APPLICATIONS

The present application claims priority to U.S. Prov. Pat. App. No. 62/947,706 entitled “Methods And Devices For Delivering Liquid Therapeutic Agents In Solid Tumors” filed Dec. 13, 2019, which is hereby incorporated by reference in its entirety.

### FIELD

The present disclosure relates generally to methods and devices for delivering liquid therapeutic agents in solid tumors.

### BACKGROUND

Treatment of a patient with a solid tumor can include delivering a liquid therapeutic agent to the solid tumor. A needle can be used to inject the liquid therapeutic agent into the solid tumor. However, backflow or reflux of the liquid therapeutic agent during the delivery process can prevent the desired amount of liquid therapeutic agent from being delivered into the tumor. The amount of liquid therapeutic agent that is in fact delivered to the solid tumor may be inadequate to effectively provide treatment and/or the liquid therapeutic agent may be inaccurately deemed to be ineffective and cause a delay or an unneeded change in the patient's treatment plan.

Additionally, backflow or reflux of the liquid therapeutic agent during delivery may require more than one delivery of the liquid therapeutic agent in order for the desired amount of the liquid therapeutic agent to be delivered. However, multiple deliveries can cause various harmful effects, such as increased costs by requiring more liquid therapeutic agent to be bought and be on hand, increased amount of patient tissue trauma, increased duration of a treatment procedure, and/or increased number of treatment procedures. It also may not be apparent to even experienced medical professionals that backflow or reflux of the liquid therapeutic agent occurred during the delivery process, so delivery of the desired amount of liquid therapeutic agent may be believed to have been successful when in fact the desired amount of liquid therapeutic agent was not delivered into the solid tumor.

Accordingly, there remains a need for delivering liquid therapeutic agents in solid tumors.

### SUMMARY

In general, methods and devices for delivering liquid therapeutic agents in solid tumors are provided.

In one aspect, a medical device is provided. In one embodiment, the medical device includes a delivery device configured to deliver a liquid therapeutic agent in a solid tumor. The delivery device includes a first elongate tubular shaft, a second elongate tubular shaft slidably disposed concentrically within the first elongate tubular shaft, and a third elongate tubular shaft slidably disposed concentrically within the second elongate tubular shaft. The third elongate tubular shaft is configured to penetrate into the solid tumor and is configured to deliver the therapeutic agent therethrough and into the solid tumor. The second elongate tubular shaft has a plurality of arms extending distally therefrom. Each of the arms has a distal tip configured to penetrate into the solid tumor. Each of the arms is biased to an expanded configuration in which the arms are expanded radially outward relative to a common longitudinal axis defined by the first, second, and third elongate tubular shafts. The first elongate tubular shaft is configured to constrain the arms therein.

The medical device can have any number of variations. For example, the second elongate tubular shaft can be configured to be advanced distally out of the first elongate tubular shaft and thereby cause the arms to automatically move from a substantially straight configuration to the expanded configuration. In at least some embodiments, the third elongate tubular shaft can be configured to be advanced distally out of the first and second elongate tubular shafts and into the solid tumor, and the second elongate tubular shaft can be configured to be advanced distally out of the first elongate tubular shaft after the advancement of the first elongate tubular shaft out of the second and third elongate tubular shafts to allow the distal tips of the arms to penetrate into the solid tumor. The first elongate tubular shaft can be configured to be advanced distally over the second and third elongate tubular shafts after the advancement of the second elongate tubular shaft out of the first elongate tubular shaft. The distal advancement of the first elongate tubular shaft can be configured to compress the arms radially inward toward a common longitudinal axis defined by the first, second, and third elongate tubular shafts. After the distal advancement of the first elongate tubular shaft the first elongate tubular shaft can be configured to move proximally relative to the second and third elongate tubular shafts, after the proximal movement of the first elongate tubular shaft the second elongate tubular shaft can be configured to move proximally relative to the first and third elongate tubular shafts, and after the proximal movement of the second elongate tubular shaft the third elongate tubular shaft can be configured to move proximally relative to the first and second elongate tubular shafts.

For another example, the arms can be biased to the expanded configuration and can be configured to automatically move from a substantially straight configuration to the expanded configuration in response to being advanced distally out of the first elongate tubular shaft.

For another example, the third elongate tubular shaft can have a sharp distal edge. For yet another example, the third elongate tubular shaft can be a hypodermic needle. For still another example, the third elongate tubular shaft can have a textured outer surface along at least a partial longitudinal length thereof, and the arms can each have a textured inner surface configured to face the textured outer surface of the third elongate tubular shaft at least when the third elongate tubular shaft is penetrated into the solid tumor. For another example, the arms constrained in the first elongate tubular shaft can each have a longitudinal axis substantially parallel to the common longitudinal axis defined by the first, second, and third elongate tubular shafts. For still another example, the arms constrained in the first elongate tubular shaft can each have a longitudinal axis that is transverse to the common longitudinal axis defined by the first, second, and third elongate tubular shafts. For yet another example, the first elongate tubular shaft can be rigid along its entire longitudinal length. For another example, the first elongate tubular shaft can be rigid along a proximal portion thereof, can be flexible along a distal portion thereof, and the first elongate tubular shaft can be configured to constrain the flexible arms within the distal portion. For yet another example, the distal tip of each of the arms can be sharp. For still another example, the arms can each be integrally formed with the second elongate tubular shaft. For another example, the arms can each be non-integral with the second elongate tubular shaft and are non-removably attached thereto. For still another example, the liquid therapeutic agent can include daratumumab, nivolumab, pembrolizumab, or ipilimumab. For another example, the solid tumor can be a melanoma, a lung cancer, a squamous non-small cell lung cancer (NSCLC), a non-squamous NSCLC, a colorectal cancer, a prostate cancer, a castration-resistant prostate cancer, a stomach cancer, an ovarian cancer, a gastric cancer, a liver cancer, a pancreatic cancer, a thyroid cancer, a squamous cell carcinoma of the head and neck, a carcinoma of the esophagus or gastrointestinal tract, a breast cancer, a fallopian tube cancer, a brain cancer, an urethral cancer, a genitourinary cancer, an endometriosis, a cervical cancer, or a metastatic lesion of cancer.

For another example, a medical system can include the delivery device and the liquid therapeutic agent configured to be delivered through the third elongate tubular shaft and into the solid tumor. In at least some embodiments, the liquid therapeutic agent can include daratumumab.

In another embodiment, a medical device includes a first elongate tubular shaft, a second elongate tubular shaft disposed within the first elongate tubular shaft, and a third elongate tubular shaft disposed within the second elongate tubular shaft. The second elongate tubular shaft includes a plurality of arms at a distal end thereof. The second elongate tubular shaft is configured to move from a first configuration, in which the arms are constrained within the first elongate tubular shaft, to a second configuration, in which distal tips of the arms are located outside of the first elongate tubular shaft and the arms bend radially outward. The third elongate tubular shaft is configured to move distally relative to the first and second elongate tubular shafts. The third elongate tubular shaft is configured to penetrate into a solid tumor and deliver a liquid therapeutic agent through an inner passageway of the third elongate tubular shaft and into the solid tumor.

The medical device can vary in any number of ways. For example, the arms can be biased to be bent radially outward. For another example, the first elongate tubular shaft can define a first longitudinal axis, the second elongate tubular shaft can define a second longitudinal axis that is coaxial with the first longitudinal axis, and the third elongate tubular shaft can define a third longitudinal axis that is coaxial with the first and second longitudinal axes.

For still another example, the second elongate tubular shaft can be configured to move distally relative to the first elongate shaft and thereby cause the second elongate tubular shaft to move from the first configuration to the second configuration. For another example, the first elongate tubular shaft can be configured to move proximally relative to the second elongate shaft and thereby cause the second elongate tubular shaft to move from the first configuration to the second configuration.

For yet another example, the third elongate tubular shaft can be configured to move distally relative to the first and second elongate tubular shafts before the second elongate shaft moves from the first configuration to the second configuration. In at least some embodiments, the second elongate tubular shaft moving from the first configuration to the second configuration can be configured to cause the distal tips of the arms to penetrate into the solid tumor. The first elongate tubular shaft can be configured to move distally over the second and third elongate tubular shafts after the movement of the second elongate tubular shaft from the first configuration to the second configuration. The distal movement of the first elongate tubular shaft can be configured to compress the arms radially inward. After the distal movement of the first elongate tubular shaft the first elongate tubular shaft can be configured to move proximally relative to the second and third elongate tubular shafts, after the proximal movement of the first elongate tubular shaft the second elongate tubular shaft can be configured to move proximally relative to the first and third elongate tubular shafts, and after the proximal movement of the second elongate tubular shaft the third elongate tubular shaft can be configured to move proximally relative to the first and second elongate tubular shafts.

For another example, the second elongate shaft moving from the first configuration to the second configuration can be configured to cause the distal tips of the arms to penetrate into the solid tumor. For yet another example, the third elongate tubular shaft can have a sharp distal edge. For still another example, the third elongate tubular shaft can be a hypodermic needle. For yet another example, the third elongate tubular shaft can have a textured outer surface along at least a partial longitudinal length thereof, and the arms can each have a textured inner surface configured to face the textured outer surface of the third elongate tubular shaft at least when the arms are in the second configuration. For another example, the arms with the second elongate tubular member in the first configuration can each have a longitudinal axis that is substantially parallel to a longitudinal axis of the second elongate tubular shaft. For still another example, the arms with the second elongate tubular member in the first configuration can each have a longitudinal axis that is not substantially parallel to a longitudinal axis of the second elongate tubular shaft. For another example, the first elongate tubular shaft can be rigid along its entire longitudinal length. For yet another example, the first elongate tubular shaft can be rigid along a proximal portion thereof, can be flexible along a distal portion thereof, and the arms in the first configuration can be located within the distal portion. For still another example, the distal tip of each of the arms can be sharp. For still another example, the arms can each be integrally formed with the second elongate tubular shaft. For another example, the arms can each be non-integral with the second elongate tubular shaft and are non-removably attached thereto. For another example, the liquid therapeutic agent can include daratumumab, nivolumab, pembrolizumab, or ipilimumab. For yet another example, the solid tumor can be a melanoma, a lung cancer, a squamous non-small cell lung cancer (NSCLC), a non-squamous NSCLC, a colorectal cancer, a prostate cancer, a castration-resistant prostate cancer, a stomach cancer, an ovarian cancer, a gastric cancer, a liver cancer, a pancreatic cancer, a thyroid cancer, a squamous cell carcinoma of the head and neck, a carcinoma of the esophagus or gastrointestinal tract, a breast cancer, a fallopian tube cancer, a brain cancer, an urethral cancer, a genitourinary cancer, an endometriosis, a cervical cancer, or a metastatic lesion of cancer.

For another example, a medical system can include the first, second, and third elongate tubular shafts and can include the liquid therapeutic agent configured to be delivered through the third elongate tubular shaft and into the solid tumor. In at least some embodiments, the liquid therapeutic agent can include daratumumab.

In another aspect, a medical method is provided that in one embodiment includes moving a distal end of a third elongate tubular shaft out of first and second elongate tubular shafts disposed around the third elongate tubular shaft such that a distal end of the third elongate tubular shaft penetrates into a solid tumor. The medical method also includes causing a plurality of arms of the second elongate tubular shaft to each penetrate into the solid tumor and moving the first elongate tubular shaft toward the solid tumor relative to the second and third elongate tubular shafts. The medical method also includes after moving the distal end of the third elongate tubular shaft, after causing the arms to each penetrate into the solid tumor, and after moving the first elongate tubular shaft toward the solid tumor, delivering a liquid therapeutic agent through the third elongate tubular shaft and into the solid tumor.

The medical method can vary in any number of ways. For example, causing the arms to each penetrate into the solid tumor can include moving the second elongate tubular shaft relative to the first elongate tubular shaft, and the movement of the second elongate tubular shaft relative to the first elongate tubular shaft can cause the arms to automatically move radially outward. In at least some embodiments, before the second elongate tubular shaft is moved relative to the first elongate tubular shaft, the first elongate tubular shaft can be constraining the arms within the first elongate tubular shaft.

For yet another example, causing the arms to each penetrate into the solid tumor can include moving the first elongate tubular shaft relative to the second elongate tubular shaft.

For another example, the arms penetrating into the solid tumor can include a sharp distal tip of each of the arms penetrating into the solid tumor. For yet another example, the distal end of the third elongate tubular shaft can have a sharp edge that penetrates into the solid tumor. For still another example, the third elongate tubular shaft can be a hypodermic needle. For yet another example, the second elongate tubular shaft can be disposed concentrically within the first elongate tubular shaft, and the third elongate tubular shaft can be disposed concentrically within the second elongate tubular shaft. For another example, moving the first elongate tubular shaft toward the solid tumor can compress the arms radially inward.

For yet another example, after delivering the liquid therapeutic agent, the medical method can include moving the first elongate tubular shaft proximally relative to the second and third elongate tubular shafts, after the proximal movement of the first elongate tubular shaft moving the second elongate tubular shaft proximally relative to the first and third elongate tubular shafts, and after the proximal movement of the second elongate tubular shaft moving the third elongate tubular shaft proximally relative to the first and second elongate tubular shafts. In at least some embodiments, moving the second elongate tubular shaft proximally relative to the first and third elongate tubular shafts can cause a distal portion of the first elongate tubular shaft to flex.

For still another example, the medical method can include advancing the first, second, and third elongate tubular shafts as a unit into a body of the patient.

For yet another example, with the distal end of the third elongate tubular shaft penetrated into the solid tumor and with the arms penetrated into the solid tumor, a textured outer surface of the third elongate tubular shaft can be aligned with a textured inner surface of each of the arms. In at least some embodiments, distal movement of the second elongate tubular shaft relative to the first elongate tubular shaft can cause the textured outer surface of the third elongate tubular shaft to become aligned with the textured inner surface of each of the arms.

For still another example, the first elongate tubular shaft can be rigid along its entire longitudinal length. For another example, the liquid therapeutic agent can include daratumumab, nivolumab, pembrolizumab, or ipilimumab. For yet another example, the solid tumor can be a melanoma, a lung cancer, a squamous non-small cell lung cancer (NSCLC), a non-squamous NSCLC, a colorectal cancer, a prostate cancer, a castration-resistant prostate cancer, a stomach cancer, an ovarian cancer, a gastric cancer, a liver cancer, a pancreatic cancer, a thyroid cancer, a squamous cell carcinoma of the head and neck, a carcinoma of the esophagus or gastrointestinal tract, a breast cancer, a fallopian tube cancer, a brain cancer, an urethral cancer, a genitourinary cancer, an endometriosis, a cervical cancer, or a metastatic lesion of cancer.

### BRIEF DESCRIPTION OF DRAWINGS

The present invention is described by way of reference to the accompanying figures which are as follows:

FIG. 1 is a side partially transparent and cross-sectional schematic view of one embodiment of a delivery device in a first configuration;

FIG. 2 is a side schematic view of a distal portion of the delivery device of FIG. 1 in another configuration;

FIG. 3 is a side partially transparent and cross-sectional schematic view of another embodiment of a delivery device in a first configuration;

FIG. 4 is a side schematic view of a distal portion of the delivery device of FIG. 3 in another configuration;

FIG. 5 is a side view of yet another embodiment of a delivery device in a position between a first configuration and a second configuration;

FIG. 6 is a side view of the delivery device of FIG. 5 in the second configuration;

FIG. 7 is a side view of the delivery device of FIG. 6 in a position between the second configuration and a third configuration;

FIG. 8 is a side view of the delivery device of FIG. 7 in the third configuration;

FIG. 9 is a side view of the delivery device of FIG. 8 in a position between the third configuration and a fourth configuration;

FIG. 10 is a side view of the delivery device of FIG. 7 in the fourth configuration;

FIG. 11 is a perspective view of another embodiment of a handle of a delivery device;

FIG. 12 is a schematic, partially transparent view of the delivery device of FIG. 2 positioned relative to soft tissue;

FIG. 13 is a schematic, partially transparent view of the delivery device of FIG. 12 positioned in another configuration relative to the soft tissue; and

FIG. 14 is a schematic, partially transparent view of the delivery device of FIG. 13 delivering a liquid therapeutic agent to the soft tissue.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary methods and devices for delivering liquid therapeutic agents in solid tumors are provided. In general, a delivery device configured to deliver a liquid therapeutic agent into a solid tumor or other soft tissue can be configured to compress tissue and seal any fluid (liquid or gas) gaps around the delivery device during the delivery of the liquid therapeutic agent. The liquid therapeutic agent can escape from the solid tumor or other soft tissue through fluid gaps surrounding the delivery device, e.g., because internal pressure built up within the solid tumor or other soft tissue can cause the liquid therapeutic agent to be expelled from the solid tumor or other soft tissue during the delivery of the liquid therapeutic agents. Therefore, sealing any fluid gaps around the delivery device may help retain the liquid therapeutic agent in the solid tumor or other soft tissue instead of backflow or reflux occurring where the liquid therapeutic agent is delivered to the solid tumor or other soft tissue and then escapes through the fluid gap(s) during the delivery process before the liquid therapeutic agent can provide any therapeutic treatment. In an exemplary embodiment, the delivery device includes three elongate tubular shafts (also referred to herein as “elongate tubes”) configured to move longitudinally relative to one another. The elongate tubular shafts are configured to cooperate with one another to deliver the liquid therapeutic agent through a passageway of the delivery device to the soft tissue and seal any fluid gaps around the delivery device while the liquid therapeutic agent is delivered into the solid tumor or other soft tissue. The elongate tubular shafts are concentric with one another, which may facilitate smooth and predictable movement of the elongate tubular shafts relative to one another and/or ease movement of the delivery device to and from a target area in a body of a patient to the solid tumor or other soft tissue since the multiple elongate tubular shafts can be moved as a unit.

In an exemplary embodiment, an inner one of the elongate tubes includes a needle configured to penetrate into the solid tumor or other soft tissue and deliver the liquid therapeutic agent therethrough into the solid tumor or other soft tissue, an outer one of the elongate tubes includes a sheath, and an intermediate one of the elongate tubes disposed between the inner and outer elongate tubes includes a plurality of arms at a distal end thereof that have distal tips configured to be in the solid tumor or other soft tissue during the delivery of the liquid therapeutic agent. The inner elongate tube penetrating into the solid tumor or other soft tissue and the arms being in the soft tissue may help prevent any movement of the solid tumor or other soft tissue relative to the inner elongate tube or arms and any movement of the inner elongate tube or arms relative to the solid tumor or other soft tissue during delivery of the liquid therapeutic agent, which may help ensure that the entire desired amount of the liquid therapeutic agent is delivered into the solid tumor or other soft tissue. Because the intermediate elongate tube surrounds the inner elongate tube, the arms of the intermediate elongate tube are arranged radially around the inner elongate tube. The arms penetrating into the solid tumor or other soft tissue around the needle penetrated into the solid tumor or other soft tissue may thus provide a barrier around the needle and help to seal any fluid gaps surrounding the needle. The sheath is configured to provide protection to the inner and intermediate tubes during advancement of the delivery device into and removal of the delivery device from a body of a patient. Also, the sheath is configured to compress in a radially inward direction the arms that are penetrated into the solid tumor or other soft tissue, which may further help seal any fluid gaps surrounding the needle during delivery of the liquid therapeutic agent into the solid tumor or other soft tissue.

In general, a liquid therapeutic agent is a material configured to be administered to a patient in liquid form to produce a beneficial medical-related effect, e.g., to help treat a medical affliction of the patient. Delivering a liquid therapeutic agent into a solid tumor or other soft tissue, instead of applying the liquid therapeutic agent (or other type of therapeutic agent) to a surface of the solid tumor or other soft tissue or delivering the liquid therapeutic agent (or other type of therapeutic agent) to a location in a patient's body away from the soft tissue and allowing the therapeutic agent to flow through the patient's circulatory system to reach the solid tumor or other soft tissue, can be desirable for a variety of reasons, e.g., because the therapeutic agent may risk side effect(s) such as tissue irritation, burning sensation, etc. that are desirable to limit to as localized an area as possible, the medical affliction being treated at the solid tumor or other soft tissue poses high danger to the patient such that immediate, localized treatment at the site of the soft tissue may provide faster treatment, to limit adverse effects of the therapeutic agent on healthy tissue by delivering the therapeutic agent directly into unhealthy tissue, a liquid is able to be injected directly into the soft tissue for treatment thereof, etc.

In an exemplary embodiment, the liquid therapeutic agent is configured to treat a solid tumor. Examples of liquid therapeutic agents configured to treat a solid tumor include Darzalex® (daratumumab), Opdivo® (nivolumab), Keytruda® (pembrolizumab), and Yervoy® (ipilimumab).

In an exemplary embodiment, the soft tissue to which the delivery device is configured to deliver the liquid therapeutic agent is a solid tumor. Examples of solid tumors include a melanoma, a lung cancer, a squamous non-small cell lung cancer (NSCLC), a non-squamous NSCLC, a colorectal cancer, a prostate cancer, a castration-resistant prostate cancer, a stomach cancer, an ovarian cancer, a gastric cancer, a liver cancer, a pancreatic cancer, a thyroid cancer, a squamous cell carcinoma of the head and neck, a carcinoma of the esophagus or gastrointestinal tract, a breast cancer, a fallopian tube cancer, a brain cancer, an urethral cancer, a genitourinary cancer, an endometriosis, a cervical cancer, and a metastatic lesion of cancer. Examples of cancers in which a patient may have a solid tumor configured to have a liquid therapeutic agent delivered thereto as described herein include bladder, brain, breast, bone, cervical, colon, esophageal, kidney, liver, lung, ovarian, pancreatic, proximal or distal bile duct, prostate, skin, stomach, thyroid, and uterine cancer. In at least some embodiments, the patient has a metastatic cancer.

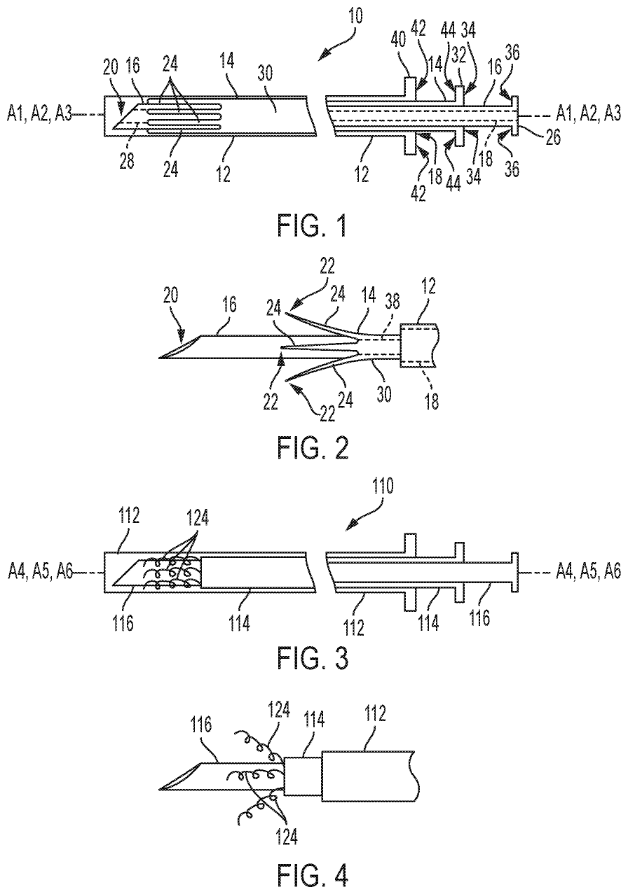

FIG. 1 illustrates one embodiment of a delivery device 10 configured to deliver a liquid therapeutic agent into a soft tissue such as a solid tumor. The delivery device 10 includes a first, outer elongate tubular shaft 12, a second, intermediate elongate tubular shaft 14 disposed within the first elongate tubular shaft 12, and a third, inner elongate tubular shaft 16 disposed within the second elongate tubular shaft 14. The elongate tubular shafts 12, 14, 16 are concentric with one another with a first longitudinal axis Al defined by the first elongate tubular shaft 12, a second longitudinal axis A2 defined by the second elongate tubular shaft 14, and a third longitudinal axis A3 defined by the third elongate tubular shaft 16 being coaxial with one another. As discussed further below, the elongate tubular shafts 12, 14, 16 are configured to move longitudinally relative to one another, e.g., to be independently movable along their respective longitudinal axes A1, A2, A3, to facilitate the delivery of the liquid therapeutic agent in the soft tissue.

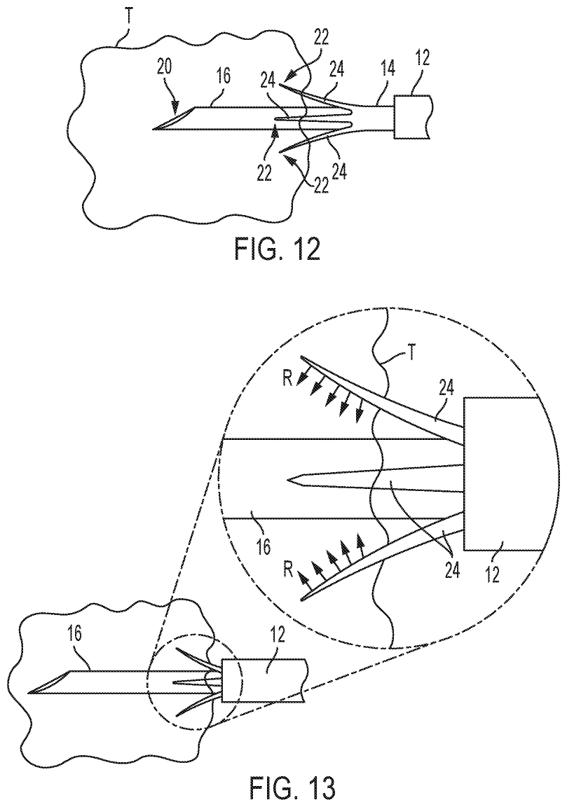

The delivery device 10 is configured to move between a first, retracted configuration, shown in FIG. 1, and a second, partially advanced configuration. The delivery device 10 is also configured to move between the second configuration and a third, advanced configuration, which is illustrated in FIG. 2 (and in FIG. 10). The delivery device 10 is also configured to move between the third configuration and a fourth configuration (illustrated in FIGS. 12 and 13).

The delivery device 10 in the first configuration is configured to be introduced into a body of a patient and advanced in the patient's body to a target soft tissue intended to have the liquid therapeutic agent delivered thereto by the delivery device 10. The delivery device 10 is also configured to be removed from the patient's body in the first configuration. In the first configuration, a distal end of the second tube 14 and a distal end of the third tube 16 are disposed within an inner lumen 18 of the first tube 12. The first tube 12 may thus help prevent an edge 20 at the distal end of the third tube 16 and distal tips 22 of arms 24 of the second tube 14 from causing damage to tissue and/or other matter during movement of the delivery device 10 in the patient's body. As in this illustrated embodiment, the edge 20 is sharp (e.g., is a beveled edge), and each of the distal tips 22 is sharp, so the first tube 12 in the first configuration may help prevent any of the sharp edge 20 and sharp distal tips 22 from inadvertently cutting tissue and/or other matter during device 10 movement. Also in the first configuration, the arms 24 of the second tube 14 are in a constrained configuration.

In the second configuration, the third tube 16 is configured to have been penetrated into target soft tissue prior to delivery of the liquid therapeutic agent into the soft tissue. In the third configuration, the distal end of the third tube 16 including the sharp edge 20 is located distal to and outside of the first tube 12, e.g., outside the inner lumen 18 of the first tube 12. The arms 24 are still in the constrained configuration.

In the third configuration, the distal end of the second tube 14, including the arms' sharp distal tips 22, and the distal end of the third tube 16, including the sharp edge 20, are located distal to and outside of the first tube 12, e.g., outside the inner lumen 18 of the first tube 12. In the third configuration, the third tube 16 and the arms 24 of the second tube 14 are configured to have each been penetrated into target soft tissue prior to delivery of the liquid therapeutic agent into the soft tissue. Also, in the third configuration, the arms 24 of the second tube 14 are in an expanded configuration in which the arms 24 are expanded radially outward. The arms 24 are biased to the expanded configuration and are configured to move automatically from the constrained configuration to the expanded configuration in response to the arms 24 being moved outside of the first tube's inner lumen 18 to be located distally beyond the first tube 12.

With the delivery device 10 in the fourth configuration, the distal end of the second tube 14, including the arms' sharp distal tips 22, and the distal end of the third tube 16, including the sharp edge 20, are located distal to and outside of the first tube 12. The delivery device 10 in the fourth configuration is configured to deliver the liquid therapeutic agent into the soft tissue. Also, in the fourth configuration, the arms 24 of the second tube 14 are in a compressed configuration in which the first tube 12 is compressing the arms 24 in a radially inward direction. As discussed further below, the first tube 12 is configured to move distally relative to the second tube 14 and thereby cause the arms 24 to move from the expanded configuration to the compressed configuration.

The delivery device 10 is configured to move from the fourth configuration to the third configuration, from the third configuration to the second configuration, and from the second configuration to the first configuration. The delivery device 10 moving back to the first configuration after being in the second, third, and fourth configurations allows the delivery device 10 to be removed from the patient's body in the first configuration.

The first, second, and third elongate tubular shafts 12, 14, 16 can have a variety of configurations. The third elongate tubular shaft 16 is configured to penetrate into soft tissue. The sharp edge 20 at the distal end of the third tube 16 is configured to facilitate the third tube's penetration into the soft tissue. The sharp edge 20 is angled and defines a point in this illustrated embodiment, which may help the third tube 16 pierce into the soft tissue and be advanced in the soft tissue with a minimal amount of tissue trauma. In an exemplary embodiment, the third elongate tubular shaft 16 is a needle, such as a hypodermic needle.

The third tube 16 includes a handle 26 at a proximal end thereof. The handle 26 is configured to facilitate user manipulation of the third tube 16, although the handle 26 in some embodiments can be manipulated by a robotic surgical system. The third tube 16 is configured to move longitudinally relative to the first and second tubes 12, 14, as mentioned above. The third tube's handle 26 is configured to be manipulated to cause the longitudinal movement of the third tube 16, e.g., moved in a proximal direction to cause proximal movement of the third tube 16 and moved in a distal direction to cause distal movement of the third tube 16.

The third tube 16 includes an inner lumen 28 extending longitudinally therethrough. A liquid therapeutic agent is configured to be passed through the inner lumen 28 and out a distal opening thereof at the third tube's distal end to deliver the liquid therapeutic agent into the soft tissue in which the third tube 16 is penetrated.

In some embodiments, the third tube 16 has a textured outer surface along at least a partial longitudinal length thereof, e.g., at least along a distal portion of the third tube 16. The textured outer surface of the third elongate tubular shaft 16 is configured to engage soft tissue when the third elongate tubular shaft 16 is penetrated into the soft tissue, e.g., when the delivery device 10 is in the second, third, and fourth configurations. The textured outer surface of the third tube 16 is configured to increase friction between the third tube 16 and the soft tissue to help prevent any movement of the soft tissue relative to the third tube 16 and any movement of the third tube 16 relative to the soft tissue, which may help ensure that the entire desired amount of the liquid therapeutic agent is delivered into the soft tissue and/or that any fluid gaps around the delivery device 10 (e.g., around the third tube 16) are sealed.

The second elongate tubular shaft 14 includes a tubular body 30 with the plurality of arms 24 extending distally therefrom. The arms 24 are each configured to penetrate into soft tissue. The sharp tips 22 of the arms 24 are pointed ends of the arms 24 configured to facilitate the arms' penetration into the soft tissue.

As shown in FIG. 1, with the delivery device 10 in the first configuration, the arms 24 are contained within the first tube 12 with an inner surface of the first tube 12 urging the arms 24 into the constrained configuration. As mentioned above, the arms 24 are also in the constrained configuration with the delivery device 10 in the second configuration. In this illustrated embodiment, the arms 24 are each substantially straight in the constrained configuration such that their respective longitudinal axes are substantially parallel to the longitudinal axis A2 of the second tube 14 and hence to the longitudinal axes A1, A3 of the first and third tubes 12, 16 that are coaxial with the second tube's longitudinal axis A2. A person skilled in the art will appreciate that the arms 24 may not be precisely straight but nevertheless be considered to be substantially straight due to any one or more factors such as manufacturing tolerances and sensitivity of measurement equipment. Similarly, a person skilled in the art will appreciate that the longitudinal axes of the arms 24 may not be precisely parallel to the longitudinal axes A1, A2, A3 but nevertheless be considered to be substantially parallel to the longitudinal axes A1, A2, A3 due to any one or more factors such as manufacturing tolerances and sensitivity of measurement equipment.

As shown in FIG. 2, with the delivery device 10 in the third configuration, the arms 24 are not fully contained within the first tube 12. The second tube 14 exiting the first tube 12, e.g., exiting the first tube's inner lumen 18, is configured to cause the arms 24 to move automatically from the constrained configuration to the expanded configuration in which the arms 24 are expanded radially outward from their position in the constrained configuration and expanded radially outward relative to the second tube's longitudinal axis A2 and hence to the longitudinal axes A1, A3 of the first and third tubes 12, 14 that are coaxial with the second tube's longitudinal axis A2. In an exemplary embodiment, the arms 24 are formed of a shape memory material (Nitinol, etc.) to facilitate the arms' automatic movement from the constrained configuration to the expanded configuration. The arms 24 being formed of a shape memory material allows the arms 24 to be thermoset in the expanded configuration, which may help the arms 24 stay in position relative to target tissue.

In some embodiments, the arms 24 each have a textured inner surface along at least a partial longitudinal length thereof, e.g., at least along a distal portion of the arms 24. The textured inner surfaces of the arms 24 are configured to engage soft tissue when the arms 24 are penetrated into the soft tissue, e.g., when the delivery device 10 is in the third and fourth configurations. The textured inner surfaces of the arms 24 are configured to increase friction between the arms 24 and the soft tissue to help prevent any movement of the soft tissue relative to the arms 24 and any movement of the arms 24 relative to the soft tissue, which may help ensure that the entire desired amount of the liquid therapeutic agent is delivered into the soft tissue and/or that any fluid gaps around the delivery device 10 (e.g., around the third tube 16) are sealed. The textured inner surfaces of the arms 24 can have any of a variety of forms, such as by the inner surfaces of the arms 24 being scored, having a plurality of dimples protruding therefrom, having a plurality of alternating grooves and ridges formed therein, etc.

The second tube 14 includes a handle 32 at a proximal end thereof. The handle 32 is configured to facilitate user manipulation of the second tube 14. The second tube 14 is configured to move longitudinally relative to the first and third tubes 12, 16, as mentioned above. The second tube's handle 32 is configured to be manipulated to cause the longitudinal movement of the second tube 14, e.g., moved in a proximal direction to cause proximal movement of the second tube 14 and moved in a distal direction to cause distal movement of the second tube 14. A proximal surface 34 of the second tube's handle 32 is configured to abut a distal surface 36 of the third tube's handle 26 when the delivery device 10 is in the second, third, and fourth configurations. The third handle's distal surface 36 abutting the second handle's proximal surface 34 provides a visual and tactile indicator that the third tube 16 has been advanced distally out of the first tube 12 and a far enough distance distally beyond the first and second tubes 12, 14 to allow the third tube 16 to penetrate into soft tissue.

The second tube 14 includes an inner lumen 38 extending longitudinally therethrough in which the third tube 16 is movably disposed.

The first elongate tubular shaft 12 is generally configured as a sheath. The first tube 12 includes a handle 40 at a proximal end thereof. The handle 40 is configured to facilitate manipulation of the first tube 12. The first tube 12 is configured to move longitudinally relative to the second and third tubes 14, 16, as mentioned above. The first tube's handle 40 is configured to be manipulated to cause the longitudinal movement of the first tube 12, e.g., moved in a proximal direction to cause proximal movement of the first tube 12 and moved in a distal direction to cause distal movement of the first tube 12. A proximal surface 42 of the first tube's handle 40 is configured to abut a distal surface 44 of the second tube's handle 32 when the delivery device 10 is in the third and fourth configurations. In at least some embodiments, the second handle's distal surface 44 abutting the first handle's proximal surface 42 can provide a visual and tactile indicator that the second tube 14 is located out of the first tube 12 a far enough distance distally beyond the first tube 12 and proximal to the third tube's distal end to allow the sharp tips 22 of the arms 24 to penetrate into soft tissue with the third tube 16 also penetrated into the soft tissue.

As in this illustrated embodiment, the first elongate tubular shaft 12 can be rigid along its entire longitudinal length. The first tube 12 being rigid may facilitate predictable positioning of the delivery device 10 relative to the target soft tissue.

In another embodiment, the first elongate tubular shaft 12 is rigid along a proximal portion thereof and is flexible along a distal portion thereof, e.g., by being made from a flexible material such as rubber, etc. The first tube 12 is configured to constrain the arms 24 of the second tube 14 within the flexible distal portion with the delivery device 10 in the first and second configurations. The flexible distal portion of the first tube 12 is configured to flex or stretch when the second tube 14 is being moved proximally relative to the first tube 12 to move the arms 24 proximally into the first tube 12. This flexing or stretching may help capture the arms 24 within the first tube 12 since the arms 24 are biased to expand rather than be compressed.

Each of the elongate tubular shafts 12, 14, 16 is made from a biocompatible material, such as a metal (e.g., stainless steel, titanium, etc.). The elongate tubular shafts 12, 14, 16 can each be made from the same material or made from a different material than one or both of the other elongate tubular shafts 12, 14, 16. In an exemplary embodiment, as shown in FIGS. 1 and 2, the arms 24 can be integrally formed with the tubular body 30, such as by being laser cut or otherwise cut into a distal portion of the tube 14. In other embodiments, such as the embodiment of FIGS. 3 and 4 discussed below, arms of a second elongate tubular shaft can be non-integrally formed with the second elongate tubular shaft's tubular body and can be non-removably attached thereto, e.g., by welding, by being press fit into holes formed in a distal surface of the tube, etc. The elongate tubular shafts 12, 14, 16 can each have a variety of diameters.

FIG. 3 illustrates another embodiment of a delivery device 110 configured to deliver a liquid therapeutic agent into a soft tissue such as a solid tumor. The delivery device 110 is generally configured and used similar to the delivery device 10 of FIG. 1 and includes a first, outer elongate tubular shaft 112, a second, intermediate elongate tubular shaft 114 disposed within the first elongate tubular shaft 112, and a third, inner elongate tubular shaft 116 disposed within the second elongate tubular shaft 114. FIG. 3 illustrates the delivery device 110 in its first configuration. FIG. 4 illustrates the delivery device 110 in its third configuration.

In this illustrated embodiment, unlike the arms 24 of the second tube 14 of FIG. 1, arms 124 of the second tube 114 are not substantially straight in their constrained configuration. Instead, the arms 124 have an irregular shape such that their respective longitudinal axes are not substantially parallel to a longitudinal axis A5 of the second tube 114 and hence to the longitudinal axes A4, A6 of the first and third tubes 112, 116 that are coaxial with the second tube's longitudinal axis A5. The irregular shape of the arms 124 in this illustrated embodiment is a spiral or helix shape, but the arms 124 can have another irregular shape, e.g., zig-zag shaped, V-shaped, etc. Additionally, the arms 124 in this illustrated embodiment have distal tips configured to penetrate tissue but are not sharp or pointed tips. Instead, the distal tips of the arms 124 have a small enough diameter to allow the distal tips to penetrate into target tissue. The arms 24 of the second tube 14 of FIGS. 1 and 2 could similarly have a small enough diameter or be thin enough to penetrate into target tissue without having sharp or pointed distal tips.

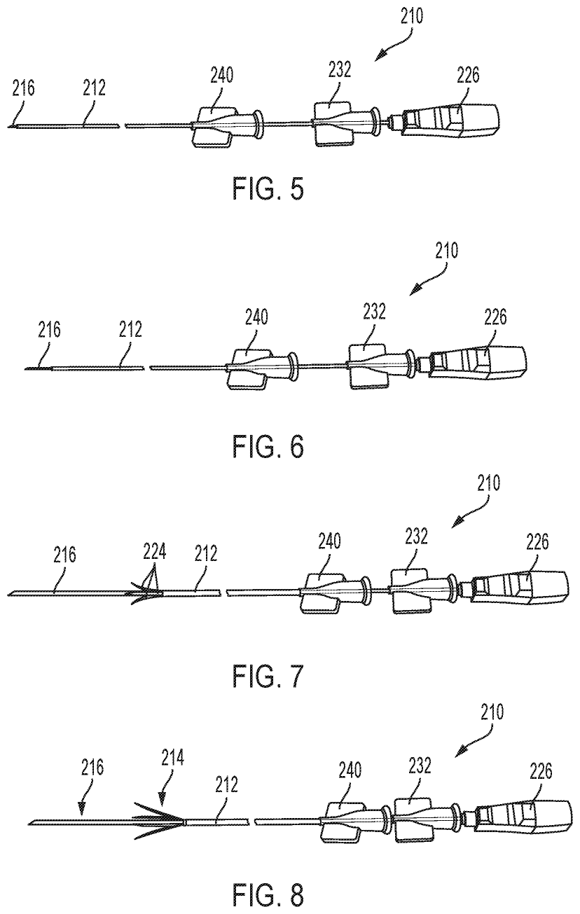

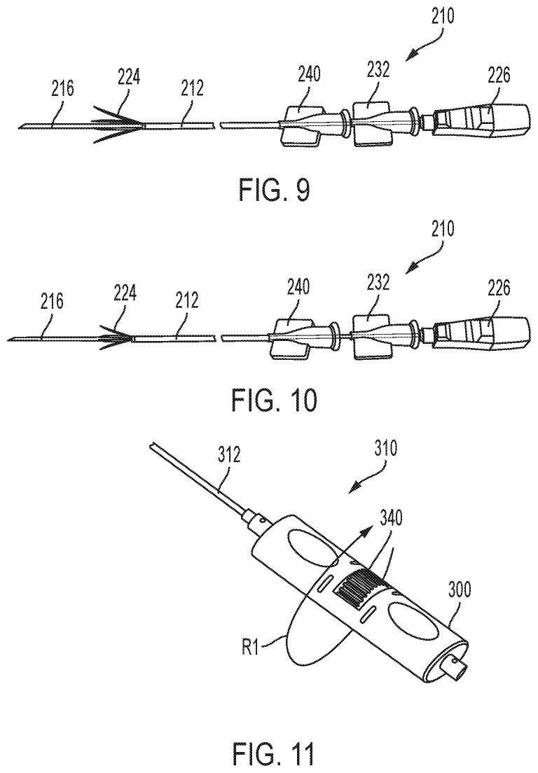

FIGS. 5-10 illustrate another embodiment of a delivery device 210 configured to deliver a liquid therapeutic agent into a soft tissue such as a solid tumor. The delivery device 210 is generally configured and used similar to the delivery device 10 of FIG. 1 and includes a first, outer elongate tubular shaft 212 including a first handle 226, a second, intermediate elongate tubular shaft 214 disposed within the first elongate tubular shaft 212 and including a second handle 232, and a third, inner elongate tubular shaft 216 disposed within the second elongate tubular shaft 214 and including a third handle 240. FIG. 5 illustrates the delivery device 210 at a position between the first configuration and the second configuration. FIG. 6 illustrates the delivery device 210 in the second configuration. FIG. 7 illustrates the delivery device 210 at a position between the second configuration and the third configuration. FIG. 8 illustrates the delivery device 210 in the third configuration. FIG. 9 illustrates the delivery device 210 at a position between the third configuration and the fourth configuration. FIG. 10 illustrates the delivery device 210 in the fourth configuration.

In this illustrated embodiment, the first elongate tubular shaft 212 is configured to be moved proximally relative to the second elongate tubular shaft 214 (and the third elongate tubular shaft 216) to allow arms 224 of the second elongate tubular shaft 214 to exit the first elongate tubular shaft 212, e.g., to move the delivery device 210 from the second configuration of FIG. 6 to the position of FIG. 7 and then to the third configuration of FIG. 8. The first handle 240 is configured to be moved proximally relative to the second elongate tubular shaft 214 (and the third elongate tubular shaft 216) to cause the proximal movement of the first elongate tubular shaft 212. The second elongate tubular shaft 214 can also be moved distally relative to the first and third elongate tubular shafts 212, 216 to further move the arms 224 out of the first elongate tubular shaft 212, e.g., by moving the second handle 232 distally.

The delivery devices 10, 110, 210 discussed above include a control mechanism, e.g., a handle, configured to be moved longitudinally to cause corresponding longitudinal movement of each handle's associated elongate tubular shaft. Each of the delivery devices 10, 110, 210 include three longitudinally translatable control mechanisms, e.g., three handles, each operatively coupled to one of the delivery device's elongate tubular shafts to allow for independent movement of each of the elongate tubular shafts. In other embodiments, a delivery device can include a control mechanism, e.g., a knob or a dial at a handle of the delivery device, configured to be rotated to cause corresponding longitudinal movement of each handle's associated elongate tubular shafts. In an exemplary embodiment, a delivery device includes three rotatable control mechanisms, e.g., three knobs, dials, etc., each operatively coupled to one of the delivery device's elongate tubular shafts to allow for independent movement of each of the elongate tubular shafts. Rotating a control mechanism to cause longitudinal movement of an elongate tubular shaft may allow for finer adjustment of elongate tubular shaft movement as compared to moving a control mechanism longitudinally to cause longitudinal movement of an elongate tubular shaft. Rotating a control mechanism to cause longitudinal movement of an elongate tubular shaft may provide a smoother, more comfortable feel to a surgeon (or other user) who is manipulating the control mechanism. Rotating a control mechanism to cause longitudinal movement of an elongate tubular shaft may facilitate user manipulation of the control mechanism with a same hand that is holding the delivery device, e.g., by allowing a thumb of the user to rotate the control mechanism while the delivery device is securely held between the user's palm and fingers.

FIG. 11 illustrates another embodiment of a delivery device 310 configured to deliver a liquid therapeutic agent into a soft tissue such as a solid tumor. The delivery device 310 is generally configured and used similar to the delivery device 10 of FIG. 1 and includes a handle 300, a first, outer elongate tubular shaft 312, a second, intermediate elongate tubular shaft (obscured in FIG. 11) disposed within the first elongate tubular shaft 312, and a third, inner elongate tubular shaft (obscured in FIG. 11) disposed within the second elongate tubular shaft. In this illustrated embodiment, each of the first, second, and third elongate tubular shafts is configured to move longitudinally in response to rotational movement of a control mechanism operatively coupled thereto.

The delivery device 310 includes a first control mechanism 340 at the handle 300 that is operatively coupled to the first elongate tubular shaft 312 via a threaded member, such as a worm gear or other gear, or other mechanism configured to translate rotational movement of the first control mechanism 340 to linear motion of the first elongate tubular shaft 312. The first control mechanism 340 is configured to rotate relative to the handle 300 to cause longitudinal movement of the first elongate tubular shaft 312 relative to the handle 300 and the first and second elongate tubular shafts. In this illustrated embodiment the first control mechanism 340 is a dial but can be another rotatable control mechanism. A knob configured to rotate relative to the handle 300 is another embodiment of a rotatable control mechanism. Yet another embodiment of a rotatable control mechanism is a first portion of the handle 300 configured to rotate relative to a remainder of the handle, similar to a kaleidoscope eyepiece being rotatable relative to a remainder of the kaleidoscope or a pepper mill head being rotatable relative to a body of the pepper mill. Rotation of the first control mechanism 340 in a first direction R1 relative to the handle 300 is configured to move the first elongate tubular shaft 312 longitudinally in a proximal direction. The first direction R1 is clockwise in this illustrated embodiment but can instead be counterclockwise. Rotation of the first control mechanism 340 in a second, opposite direction, e.g., counterclockwise, relative to the handle 300 is configured to move the first elongate tubular shaft 312 longitudinally in a distal direction.

The delivery device 310 can also include second and third control mechanisms (not shown) at the handle 300 that are operatively coupled to the second and third elongate tubular shafts, respectively. The second and third control mechanisms can be configured and used similar to that discussed above regarding the first control mechanism 340.

FIGS. 12-14 illustrate one embodiment of a method of using a delivery device as described herein. Although the method is described with respect to the delivery device 10 of FIGS. 1 and 2, other embodiments of delivery devices described herein can be similarly used.

The delivery device 10 is introduced into a body of a patient, either directly, e.g., through an incision, or indirectly, e.g., through an access device such as a trocar, cannula, or scoping device. The delivery device 10 is advanced to a target soft tissue T in the body of the patient and positioned as desired relative to and outside of the soft tissue T. Imaging equipment can be used to assist placement of the delivery device 10 in the desired position. The soft tissue T in this illustrated embodiment is a solid tumor, but as discussed above, the delivery device 10 can be used to deliver a liquid therapeutic agent to other soft tissues. In an exemplary embodiment, the delivery device 10 is in the first configuration during the introduction of the delivery device 10 into the patient's body and during the delivery device's advancement to and positioning relative to the soft tissue T.

With the delivery device 10 in the first configuration and positioned as desired relative to and outside of the soft tissue T, the third tube 16 is moved distally relative to the first and second tubes 12, 14 to penetrate the sharp edge 20 of the third tube 16 into the soft tissue T. In other words, the delivery device 10 is moved from the first configuration to the second configuration.

The distal movement of the third tube 16 can be achieved by moving the third handle 26 distally. The third handle 26 can be moved distally until the third handle's distal surface 36 abuts the second handle's proximal surface 34 which, as mentioned above, indicates that the third tube 16 has been advanced out of the first tube 12 and has advanced a sufficient distance to allow the sharp edge 20 and a distal portion of the third tube 16 to penetrate into the soft tissue T. Additionally or alternatively, the third tube's position can be verified using imaging equipment.

With the third tube 16 penetrated into the soft tissue T, e.g., with the delivery device 10 in the second configuration, the second tube 14 is moved distally relative to the first and third tubes 12, 16 to penetrate the sharp tips 22 of the arms 24 into the soft tissue T, as shown in FIG. 12. In other words, the delivery device 10 is moved from the second configuration to the third configuration. The distal movement of the second tube 14 can be achieved by moving the second handle 32 distally. The second handle 32 can be moved distally until the second handle's distal surface 44 abuts the first handle's proximal surface 42 which, as mentioned above, indicates that the second tube 14 has been advanced out of the first tube 12 and has advanced a sufficient distance to allow the sharp tips 22 and a distal portion of the arms 24 to penetrate into the soft tissue T. Additionally or alternatively, the second tube's position can be verified using imaging equipment.

With the third tube 16 and the arms 24 penetrated into the soft tissue T, e.g., with the delivery device 10 in the third configuration, the first tube 12 is moved distally relative to the second and third tubes 14, 16 to compress the arms 24 radially inward as shown by arrows R in FIG. 13 that point radially inward toward the coaxial longitudinal axes A1, A2, A3. In other words, the delivery device 10 is moved from the third configuration to the fourth configuration. As discussed herein, the arms 24 compressing radially inward may help compress the tissue T around the third tube 16 and help prevent fluid backflow or reflux. The distal movement of the first tube 12 can be achieved by moving the first handle 40 distally. In an exemplary embodiment, the first tube 12 is advanced distally until a distal surface thereof abuts the soft tissue T. The abutting of the first tube 12 against the soft tissue T can be tactilely felt by a user of the delivery device 10 and/or can be confirmed via visualization of the first tube 12 and soft tissue T.

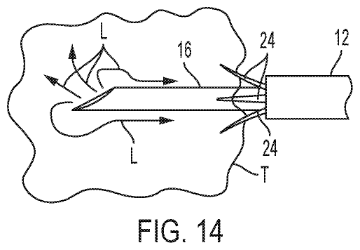

With the third tube 16 and the arms 24 penetrated into the soft tissue T and the first tube 12 compressing the arms 24 radially inward, e.g., with the delivery device 10 in the fourth configuration, a liquid therapeutic agent L is delivered through the delivery device 10, e.g., through the inner lumen 28 of the third tube 16, and into the soft tissue T, as shown in FIG. 14. As discussed herein, the second tube 14 (e.g., the arms 24 thereof) and the first tube 12 cooperate to prevent reflux or backflow of the liquid therapeutic agent L out of the soft tissue T. The liquid therapeutic agent L in this illustrated embodiment is expelled out of an open end of the third tube 16, e.g., the distal opening of the inner lumen 28, but the liquid therapeutic agent L can be delivered out of the third tube 16 in other ways in other embodiments. For example, a plurality of holes can be formed in a distal portion of the third tube 16 to allow the liquid therapeutic agent L to be delivered therethrough in addition to or instead of through an open end of the third tube 16. For another example, a distal portion of the third tube 16 can be formed of a porous material configured to allow the liquid therapeutic agent L to pass therethrough. For yet another example, the inner lumen 28 can be a plurality of lumens instead of a single lumen with the therapeutic agent L being delivered through each of the plurality of lumens.

After delivering the liquid therapeutic agent L into the soft tissue T, the delivery device 10 is removed from the patient's body. In an exemplary embodiment, the delivery device 10 is moved from the fourth configuration back to the first configuration before being removed from the patient's body. Alternatively, the delivery device 10 can be moved to a second target soft tissue and used to deliver a second liquid therapeutic agent to the second target soft tissue as discussed above. The delivery device 10 can then be removed from the patient's body or can be used to similarly deliver a liquid therapeutic agent to one or more additional target soft tissues before being removed from the patient's body.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device is described in more detail in U.S. Pat. No. 8,114,345 issued Feb. 14, 2012 and entitled “System And Method Of Sterilizing An Implantable Medical Device.”

The present disclosure has been described above by way of example only within the context of the overall disclosure provided herein. It will be appreciated that modifications within the spirit and scope of the claims may be made without departing from the overall scope of the present disclosure.

1. A medical device, comprising:
a delivery device configured to deliver a liquid therapeutic agent in a solid tumor and including a first elongate tubular shaft, a second elongate tubular shaft slidably disposed concentrically within the first elongate tubular shaft, and a third elongate tubular shaft slidably disposed concentrically within the second elongate tubular shaft; wherein:
the third elongate tubular shaft is configured to penetrate into the solid tumor and is configured to deliver the therapeutic agent therethrough and into the solid tumor;
the second elongate tubular shaft has a plurality of arms extending distally therefrom, each of the arms having a distal tip configured to penetrate into the solid tumor, and each of the arms being biased to an expanded configuration in which the arms are expanded radially outward relative to a common longitudinal axis defined by the first, second, and third elongate tubular shafts; and
the first elongate tubular shaft is configured to constrain the arms therein.

2. The device of claim 1, wherein the second elongate tubular shaft is configured to be advanced distally out of the first elongate tubular shaft and thereby cause the arms to automatically move from a substantially straight configuration to the expanded configuration.

3. The device of claim 2, wherein the third elongate tubular shaft is configured to be advanced distally out of the first and second elongate tubular shafts and into the solid tumor; and
the second elongate tubular shaft is configured to be advanced distally out of the first elongate tubular shaft after the advancement of the first elongate tubular shaft out of the second and third elongate tubular shafts to allow the distal tips of the arms to penetrate into the solid tumor.

4. The device of claim 3, wherein the first elongate tubular shaft is configured to be advanced distally over the second and third elongate tubular shafts after the advancement of the second elongate tubular shaft out of the first elongate tubular shaft.

5. The device of claim 4, wherein the distal advancement of the first elongate tubular shaft is configured to compress the arms radially inward toward a common longitudinal axis defined by the first, second, and third elongate tubular shafts.

6. The device of claim 4, wherein, after the distal advancement of the first elongate tubular shaft, the first elongate tubular shaft is configured to move proximally relative to the second and third elongate tubular shafts;
after the proximal movement of the first elongate tubular shaft, the second elongate tubular shaft is configured to move proximally relative to the first and third elongate tubular shafts; and
after the proximal movement of the second elongate tubular shaft, the third elongate tubular shaft is configured to move proximally relative to the first and second elongate tubular shafts.

7. The device of claim 1, wherein the arms are biased to the expanded configuration and are configured to automatically move from a substantially straight configuration to the expanded configuration in response to being advanced distally out of the first elongate tubular shaft.

8. The device of claim 1, wherein the third elongate tubular shaft has a sharp distal edge.

9. The device of claim 1, wherein the third elongate tubular shaft is a hypodermic needle.

10. The device of claim 1, wherein the third elongate tubular shaft has a textured outer surface along at least a partial longitudinal length thereof; and
the arms each have a textured inner surface configured to face the textured outer surface of the third elongate tubular shaft at least when the third elongate tubular shaft is penetrated into the solid tumor.

11. The device of claim 1, wherein the arms constrained in the first elongate tubular shaft each have a longitudinal axis substantially parallel to the common longitudinal axis defined by the first, second, and third elongate tubular shafts.

12. The device of claim 1, wherein the arms constrained in the first elongate tubular shaft each have a longitudinal axis that is transverse to the common longitudinal axis defined by the first, second, and third elongate tubular shafts.

13. The device of claim 1, wherein the first elongate tubular shaft is rigid along its entire longitudinal length.

14. The device of claim 1, wherein the first elongate tubular shaft is rigid along a proximal portion thereof and is flexible along a distal portion thereof, the first elongate tubular shaft being configured to constrain the flexible arms within the distal portion.

15. The device of claim 1, wherein the distal tip of each of the arms is sharp.

16. The device of claim 1, wherein the arms are each integrally formed with the second elongate tubular shaft.

17. The device of claim 1, wherein the arms are each non-integral with the second elongate tubular shaft and are non-removably attached thereto.

18. The device of claim 1, wherein the liquid therapeutic agent includes daratumumab, nivolumab, pembrolizumab, or ipilimumab.

19. The device of claim 1, wherein the solid tumor is a melanoma, a lung cancer, a squamous non-small cell lung cancer (NSCLC), a non-squamous NSCLC, a colorectal cancer, a prostate cancer, a castration-resistant prostate cancer, a stomach cancer, an ovarian cancer, a gastric cancer, a liver cancer, a pancreatic cancer, a thyroid cancer, a squamous cell carcinoma of the head and neck, a carcinoma of the esophagus or gastrointestinal tract, a breast cancer, a fallopian tube cancer, a brain cancer, an urethral cancer, a genitourinary cancer, an endometriosis, a cervical cancer, or a metastatic lesion of cancer.

20. A medical system, comprising:
the delivery device of claim 1; and
the liquid therapeutic agent configured to be delivered through the third elongate tubular shaft and into the solid tumor.

21. The system of claim 20, wherein the liquid therapeutic agent includes daratumumab.

22-63. (cancelled)
